(19) United States
(12) Patent Application Publication
(10) Pub. No.: US 2007/0178103 A1
(43) Pub. Date: Aug. 2, 2007

(54) CD19-specific immunotoxin and treatment method

(76) Inventors: Georg H. Fey, Neunkirchen a. Br. (DE); Matthias Peipp, Hamburg (DE); Michael Schwemmlein, Erlangen (DE)

Correspondence Address: PERKINS COIE LLP, P.O. BOX 2168, MENLO PARK, CA, 94026, US

(21) Appl. No.: 11/344,466

(22) Filed: Jan. 30, 2006

(51) Int. Cl. A61K 39/395; C07K 16/46

(52) U.S. Cl. 424/155.1; 424/178.1; 530/391.1

(57) ABSTRACT

An immunotoxin for use in, and a method for treating a subject having a cancer associated with malignant B-lineage cells or an autoimmune condition, are disclosed. The immunotoxin includes (a) an anti-CD19 antibody lacking an Fc fragment, (b) a modified exotoxin A protein having both Domains II and III, but lacking Domain I, and (c) a peptide linker joining the C-terminal end of the antibody to the N-terminal end of the modified exotoxin A protein. The linker is substantially resistant to extracellular cleavage. The modified exotoxin A protein may be further modified to include a C-terminal KDEL sequence (SEQ ID NO: 6) that promotes transport of the protein to the endoplasmic reticulum of cells that have taken up the immunotoxin.

### FIELD OF THE INVENTION

This invention relates to a CD-19 specific immunotoxin and treatment methods employing the immunotoxin.

### BACKGROUND OF THE INVENTION

CD19, a cell surface glycoprotein of the immunoglobulin superfamily is a potentially attractive target for antibody therapy of B-lymphoid malignancies. This antigen is absent from hematopoietic stem cells, and in healthy individuals its presence is exclusively restricted to the B-lineage and possibly some follicular dendritic cells (Scheuermann, R. et al. (1995) Leuk Lymphoma 18, 385-397). Furthermore, CD19 is not shed from the cell surface and rarely lost during neoplastic transformation (Scheuermann, 1995). The protein is expressed on most malignant B-lineage cells, including cells from patients with chronic lymphocytic leukemia (CLL), Non-Hodgkin lymphoma (NHL), and acute lymphoblastic leukemia (ALL) (Uckun, F. M. et al. (1988) Blood 71, 13-29). Importantly, CD19 is consistently expressed on B-precursor and mature B-ALLs, whereas CD20 is less frequently expressed, particularly on B-precursor ALLs (Hoelzer, D. et al. (2002) Hematology (Am Soc Hematol Educ Program), 162-192).

Immunotoxins composed of a toxin linked to an antibody specific against cell-surface antigens, including CD19, have been proposed in the treatment of various cancers. However, such immunoconjugates have been limited in their use, heretofore, by extracellular cytotoxicity problems, such as hepatotoxicity, pulmonary toxicity, and/or severe hypersensitivity reactions. Ideally, an immunotoxin for use in treating B-cell malignancies would have a reduced toxicity before being taken up into target cells, and efficient uptake and retention within target cells. The present invention is aimed at providing such an immunotoxin, and its use in treating various B-cell malignancies.

### SUMMARY OF THE INVENTION

The invention includes, in one aspect, an immunotoxin for use in treating a subject having a cancer associated with malignant B-lineage cells, such as chronic lymphocytic leukemia, Non-Hodgkin lymphoma, and acute lymphoblastic leukemia. The immunotoxin includes (a) a anti-CD19 antibody lacking an Fc fragment, (b) a modified Pseudomonas aeruginosa exotoxin A protein having both Domains II and III, but lacking Domain I, and (c) a peptide linker joining the C-terminal end of the antibody to the N-terminal end of the modified exotoxin A protein. The linker is substantially resistant to extracellular cleavage.

The exotoxin A protein may have a C-terminal KDEL sequence (SEQ ID NO: 6) that promotes transport of the protein to the endoplasmic reticulum of cells that have taken up the immunotoxin, such as the modified exotoxin A protein having the sequence identified by SEQ ID NO: 3.

The antibody may be a single-chain scFv antibody composed of a variable-region light chain coupled to a variable-region heavy chain through a glycine/serine-peptide linker.

The antibody may be coupled to the modified exotoxin A protein through a glycine/serine-peptide linker, such as the linker having the sequence identified as SEQ ID NO: 5.

In another aspect, the invention includes a method of treating a subject having a cancer associated with malignant B-lineage cells, such as chronic lymphocytic leukemia, Non-Hodgkin lymphoma, and acute lymphoblastic leukemia. The method comprises administering to the patient, a therapeutically effective amount of the above immunotoxin.

In still another aspect, the invention includes a method for treating an autoimmune disease, such as multiple sclerosis, rheumatoid arthritis, and SLE, comprising administering to the patient, a therapeutically effective amount of the above immunotoxin.

Also disclosed is a method for delivering exotoxin A (ETA) to a human subject, in the treatment of a cancer having cancer-specific cell-surface antigens. The method comprises (a) replacing Domain I of the ETA with a single-chain antibody specific against the cell-surface antigen and a peptide linker joining the C-terminal end of the antibody to the N-terminal end of the modified ETA, and (b) replacing the REDLK C-terminal sequence (SEQ ID NO: 7) of ETA with a KDEL sequence (SEQ ID NO: 6) that promotes transport of the protein to the endoplasmic reticulum. The linker is substantially resistant to extracellular cleavage.

For use in treating a subject having a cancer associated with malignant B-lineage cells, such as chronic lymphocytic leukemia, Non-Hodgkin lymphoma, and acute lymphoblastic leukemia, the single-chain antibody replacing the ETA Domain I may be an antibody specific against CD19 B-cell antigen, such as an anti-CD19 scFv antibody. The linker may include a glycine/serine-peptide linker, such as a linker having the sequence identified as SEQ ID NO: 5.

These and other objects and features of the invention will become more fully apparent when the following detailed description of the invention is read in conjunction with the accompanying drawings.

### BRIEF DESCRIPTION OF THE DRAWINGS

FIG. 1 is a schematic representation of the recombinant immunotoxin CD19-ETA′. STREP, N-terminal STREP tag; 6xHis, hexahistidine tag; V_{L} and V_{H}, variable region light and heavy chains of the CD19-specific scFv; linker, flexible linkers consisting of glycine and serine residues; Exotoxin A′, truncated Exotoxin A fragment consisting of domains II and III of the Pseudomonas toxin; KDEL (SEQ ID NO: 6), ER retention motif.

FIGS. 2A and 2B are each a graph of the number of cells versus the fluorescence intensity showing specific binding of the recombinant immunotoxin to antigen-positive cells. Cells were stained with purified CD19-ETA′ fusion protein (black) or a nonrelated scFv-ETA′ fusion protein (white) at the same concentration and analyzed by FACS. FIG. 2A shows results for CD19-positive Namalwa cells stained with CD19-ETA′. FIG. 2B shows results for CD19-negative U937 cells stained with CD19-ETA′.

FIG. 3 is a graph showing the results of how CD19-ETA′ reduces the number of viable Nalm-6 cells during 96 hrs. Nalm-6 cells were treated with PBS or CD19-ETA′ at time point 0. At the indicated time points, viable cells were counted by trypan blue exclusion. Triplicate samples were measured for each time point and standard deviations are indicated by error bars.

FIGS. 4A and 4B are graphs showing the results of how CD19-ETA′ induces cell death of CD19-positive Nalm-6 cells at low concentrations but not of CD19-negative CEM cells. Nalm-6 (FIG. 4A) and CEM cells (FIG. 4B) were treated with single doses of the indicated concentrations of CD19-ETA′ for 72 h. Aliquots of cells were evaluated for percentage of cell death by PI staining of nuclei and FACS analysis. Data points are mean values from four independent experiments and standard deviations are indicated by error bars.

FIG. 5 shows images of cells stained with Annexin V and PI after 48 h of treatment with CD19-ETA′. The results show that CD19-ETA′ induces apoptosis in CD19-positive Nalm-6 (frames A-C), Namalwa (frames D-F) and Reh cells (frames G-I). Preincubation of the cells with the parental antibody 4G7 prevents the cells from being killed by CD19-ETA′. The cells were treated with PBS alone (frames A, D and G), single doses of 500 ng/ml CD19-ETA′ alone (frames B, E, and H) or were preincubated with a molar excess of the parental CD19 antibody 4G7 (frames C, F, and I). Numbers in the upper right quadrant of each plot represent the percentage of Annexin V-positive cells.

FIGS. 6A and 6B are graphs showing the results of how CD19-ETA′ kills primary cells of two patients suffering from chronic lymphocytic leukemia (CLL) (6A and 6B). Primary CLL cells were treated with PBS (white bars), CD19-ETA′ (black bars) or a control immunotoxin CD33-ETA′ (grey bars) at time point 0. At the indicated time points, the percentage of Annexin V-positive cells was determined. Triplicate samples were measured for each time point and standard deviations are indicated by error bars.

### DETAILED DESCRIPTION OF THE INVENTION

I. Definitions

The following terms have the meaning defined herein, except when indicated otherwise.

An “anti-CD19 antibody” or “CD19-specific antibody” or “CD19 antibody” refers to an antibody that specifically recognizes the cell-surface glycoprotein of the immunoglobulin superfamily commonly referred to as CD19.

The term “antibody”, as used herein, encompasses immunoglobulin molecules comprised of four polypeptide chains, two heavy (H) chains and two light (L) chains inter-connected by disulfide bonds. Each chain consists of a variable portion, denoted V_{H} and V_{L} for variable heavy and variable light portions, respectively, and a constant region, denoted CH and CL for constant heavy and constant light portions, respectively. The CH portion contains three domains CH1, CH2, and CH3. Each variable portion is composed of three hypervariable complementarity determining regions (CDRs) and four framework regions (FRs).

The Fc fragment of an antibody refers to the crystalline fragment of an immunoglobulin which is released by, e.g., papain digestion of an immunoglobulin, and which is responsible for many of the effector functions of immunoglobulins.

An “antibody lacking an Fc fragment” refers to any of a variety of antibody fragments lacking the effector functions of the Fc fragment, and may include (i) an Fab fragment, which is a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) an Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). In particular, although the two domains of the Fv fragment, V_{L} and V_{H}, are coded for by separate genes, they can be joined by recombinant methods, by a synthetic linker that enables them to be made as a single protein chain in which the V_{L} and V_{H} regions pair to form monovalent molecules known as single chain variable fragment or scFv antibodies; see e.g., Bird et al. (1988) Science 242:423-426; and Huston et al. (1988) Proc. Natl. Acad. Sci. USA 85:5879-5883), and the term antibody lacking an Fc fragment also encompasses antibodies having this scFv format.

The term “recombinant antibody”, as used herein, is intended to include antibodies that are prepared, expressed, created or isolated by recombinant means, such as antibodies expressed using a recombinant expression vector transfected into a host cell.

A “glycine/serine” linker refers to a linear polypeptide chain composed substantially, e.g., at least 80%, and preferably entirely of glycine and serine amino acid residues.

The three-letter and one-letter amino acid abbreviations and the single-letter nucleotide base abbreviations used herein are according to established convention, as given in any standard biochemistry or molecular biology textbook.

II. Construction of the Anti-CD19 Immunotoxin

The invention includes an immunotoxin composed of (1) a CD19-specific antibody lacking an Fc fragment, e.g., a single chain Fv (scFV) antibody fragment, (2) an engineered variant of Pseudomonas Exotoxin A (ETA) having both Domains II and III, but lacking Domain I, and (3) a peptide linker joining the C-terminal end of the antibody to the N-terminal end of the modified exotoxin A protein. The linker is substantially resistant to extracellular cleavage.

A CD19-specific antibody lacking an Fc fragment may be constructed according to known methods. Where the antibody is an anti-CD19 scFv antibody, the methods detailed in Example 1 are suitable. In this example, the scFv antibody fragment may be constructed by isolating by screening a phage display library generated from RNA of the CD19 hybridoma 4G7 (Meeker, T. C., Miller, R. A., Link, M. P., Bindl, J., Warnke, R., and Levy, R. A unique human B lymphocyte antigen defined by a monoclonal antibody. Hybridoma, 3: 305-320, 1984).

As just noted, the toxin moiety of the immunotoxin of the invention is Pseudomonas Exotoxin A (ETA), specifically, a truncated version lacking domain I and containing only domains II and III. (Wels, W., Beerli, R., Hellmann, P., Schmidt, M., Marte, B. M., Kornilova, E. S., Hekele, A., Mendelsohn, J., Groner, B., and Hynes, N. E). The EGF receptor and p185erbB-2-specific single-chain antibody toxins differ in their cell-killing activity on tumor cells expressing both receptor proteins. Int J Cancer, 60: 137-144, 1995). Domain I is the binding domain for the α₂-macroglobulin receptor (CD91) present on most mammalian cells (Kounnas, M. Z., Morris, R. E., Thompson, M. R., FitzGerald, D. J., Strickland, D. K., and Saelinger, C. B. The alpha 2-macroglobulin receptor/low density lipoprotein receptor-related protein binds and internalizes Pseudomonas exotoxin A. J Biol Chem, 267:12420-12423, 1992).

Domains II and III of ETA are required for intracellular transport and carry the active center of the toxin, respectively, which inhibits protein synthesis by blocking the translation elongation factor EF-2 and causes apoptosis (Lord, J. M., Smith, D. C., and Roberts, L. M. Toxin entry: how bacterial proteins get into mammalian cells. Cell Microbiol, 1: 85-91, 1999). Consequently, the truncated variant of ETA, abbreviated ETA′, which lacks domain I is not toxic as long as it remains in the extracellular space. In addition, ETA′ can be administered with fewer side effects on vascular endothelial cells, because it has a much lower affinity to these cells than, for example, ricin A.

Replacing the domain I of ETA with an antibody fragment directed against an antigen capable of internalization, converts the ETA′ variant into a potent immunotoxin. Moreover, the modified ETA′ may be further modified to contain a C-terminal KDEL (SEQ ID NO: 6) motif, the characteristic ER retention sequence of a variety of luminal ER proteins (Munro, S. and Pelham, H. R. A C-terminal signal prevents secretion of luminal ER proteins. Cell, 48: 899-907, 1987). Further, coupling the modified ETA to the CD-19 antibody through a linker that is substantially resistant to extracellular cleavage reduces the potential for toxicity due to release of the toxin into the bloodstream before the immunotoxin reaches the target cells. As will be seen below, the immunotoxin of the present invention shows that a CD19-specific scFv fused to ETA′ is effective at very low concentrations against CD19-positive leukemia cell lines and primary cells from CLL patients, and displays exquisite antigen-specific activity.

To construct the coding sequence for the immunotoxin protein, the scFv cDNA insert from a reactive phage isolate was subcloned and fused to the coding sequence for truncated Pseudomonas Exotoxin A lacking the receptor-binding domain (Example 2). The coding sequence for the C-terminal pentapeptide REDLK (SEQ ID NO: 7), a peptide directing the retrograde transport of the authentic toxin, was replaced by the coding sequence for the KDEL-tetrapeptide (SEQ ID NO: 6), a peptide assuring proper retrograde transport of cellular proteins. This replacement was performed following published examples (Brinkmann, U., Pai, L. H., FitzGerald, D. J., Willingham, M., and Pastan, I. B3(Fv)-PE38KDEL, a single-chain immunotoxin that causes complete regression of a human carcinoma in mice. Proc Natl Acad Sci USA, 88: 8616-8620, 1991) to optimize intracellular transport to the ER. In one embodiment, the variable light and heavy chain domains (V_{L} and V_{H}) are linked by a sequence coding for a 20 amino acid synthetic linker, and given by SEQ ID NO: 4. In the same embodiment, the scFv antibody and ETA′ toxin are linked by a sequence coding for a 20 amino acid synthetic linker, and given by SEQ ID NO: 5.

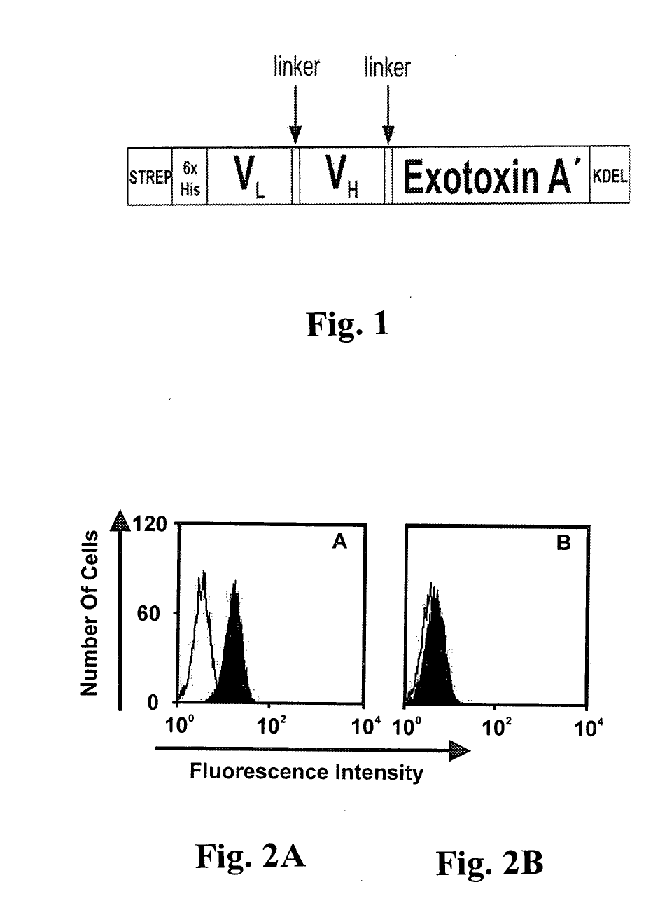

Sequences coding for a STREP-tag (WSHPQFEK, SEQ ID NO: 8) and a hexahistidine-tag were added at the N-terminus for detection and purification and a schematic representation of the resulting purified fusion protein is shown in FIG. 1. The complete coding sequence for the fusion protein is given by SEQ ID NO:1 below, and the amino acids sequence for the fusion protein, by SEQ ID NO: 2. The resulting polypeptide was expressed in E. coli and purified from periplasmic extracts by affinity chromatography using a streptactin matrix. The fusion protein of the invention which is referred to as the CD19-immunotoxin (termed CD19-ETA′) specifically reacted with the CD19-positive human Burkitt lymphoma derived cell line Namalwa as visualized by flow cytometry (see FIG. 2). The agent failed to react with CD19-negative monocytic U937-cells.

III. Characterization of an scFv-ETA′ Immunotoxin

A. Antigen-Specific Cytotoxic Activity of the Immunotoxin

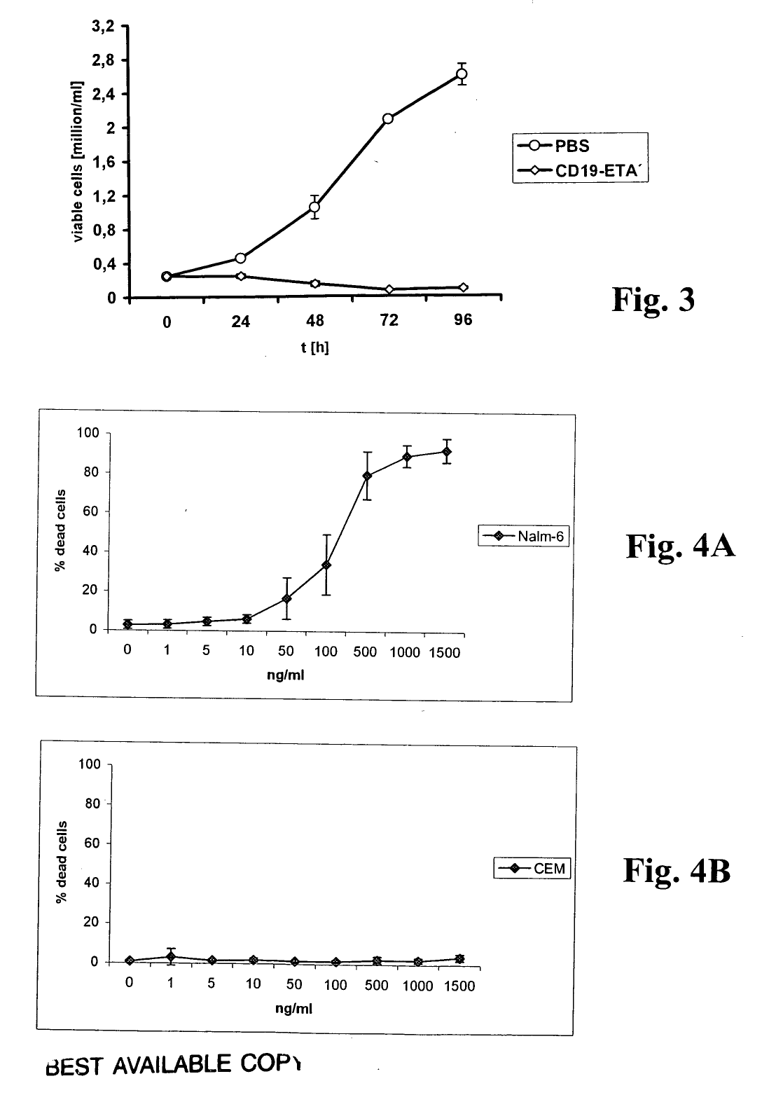

CD19-ETA′ mediated specific death of CD19-positive Nalm-6 cells, but failed to eliminate CD19-negative CEM cells, as evidenced by counting viable cells every 24 h for 96 h (FIG. 3), and measurement of nuclear DNA content after 72 h of treatment, using propidium iodide (PI) staining and flow cytometry with the results being graphed in FIG. 4. Maximum lysis of Nalm-6 cells within 72 h was achieved with single doses of 1 μg/ml (14 nM). Same concentrations of the immunotoxin failed to kill antigen-negative CEM cells. Thus, these results show that CD19-ETA′ acts in a highly antigen-specific manner and is effective for cultured malignant cells in the low nanomolar concentration range. The results demonstrate that the toxin is highly specific for cells expressing surface antigen CD19, and that selective cell killing is effective in the nM range of immunotoxin.

B. CD19-ETA′ Eliminates Cells by Apoptosis.

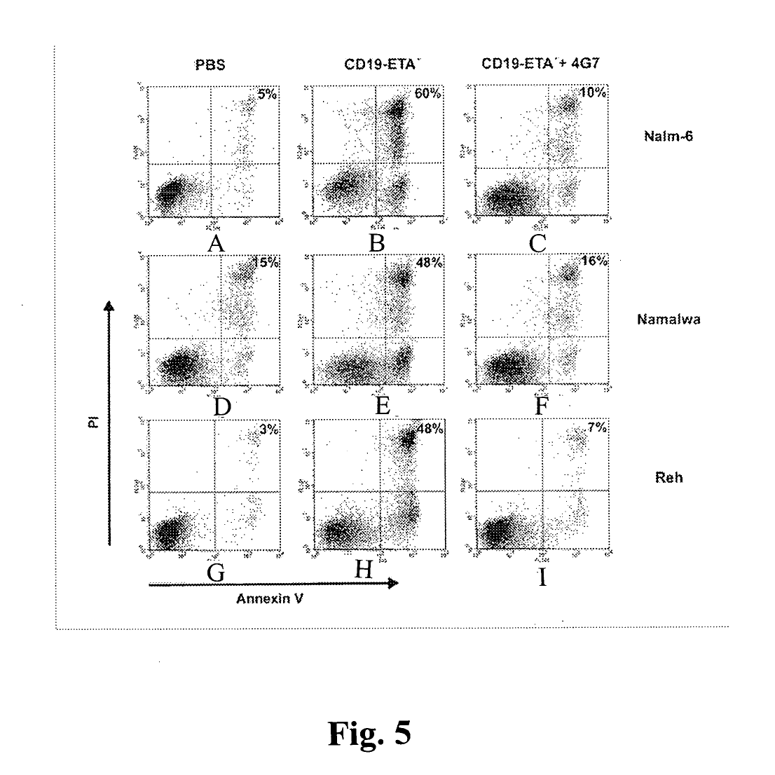

To investigate whether death induced by the agent occurred via apoptosis or other cellular routes to elimination, apoptosis was specifically measured by Annexin V and PI staining. This method of Annexin V and PI staining provides independent evidence for cell death by apoptosis beyond the method of counting cells with SubG₁-DNA content presented above (FIG. 4). CD19-ETA′ induced apoptosis of antigen-positive human B cell precursor leukemia derived cell lines Nalm-6 and Reh, and of human Burkitt lymphoma derived Namalwa cells. For comparison, cell death was blocked by pretreatment with excess concentrations of the parental CD19 antibody 4G7 (FIG. 5). These results confirm the ability of CD19-ETA′ to kill target cells by apoptosis in a highly antigen-specific manner for different CD19-positive tumor-derived human cell lines representing different disease entities.

C. CD19-ETA′ Induces Cell Death of Primary CLL Cells

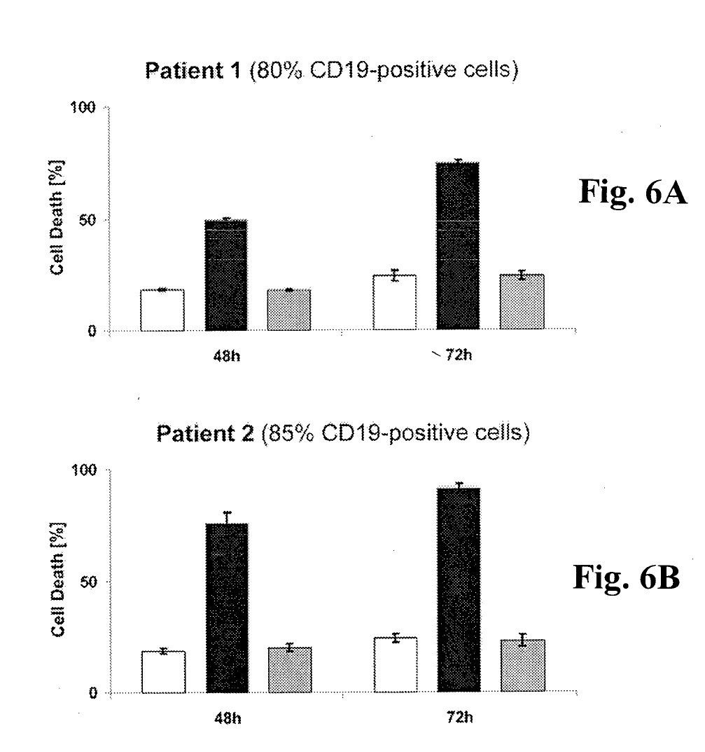

CD19-ETA′ also mediated death of primary cells from two patients suffering from CLL (FIG. 6). The induction of cell death by the CD19-ETA′ immunotoxin was antigen-specific because a control immunotoxin directed against an antigen not expressed on the CLL cells was not able to kill the cells.

IV. Therapeutic Method

The immunotoxin of the invention is useful in treating a human subject having a cancer associated with malignant B-lineage cells, such as chronic lymphocytic leukemia, Non-Hodgkin lymphoma, and acute lymphoblastic leukemia, as evidenced by (i) the ability of the immunotoxin to exhibit its cytotoxic effects in the concentration range of ng/ml, (ii) the cytolysis by the immunotoxin is highly antigen-specific, and (iii) immunotoxin induced cell death occurs by apoptosis as demonstrated by Annexin V staining.

In the immunotherapy approach, a patient diagnosed with a cancer associated with malignant B-lineage cells is treated by administration of the immunotoxin, preferably administered by IV injection in a suitable physiological carrier. The immunotoxin dose is preferably 1 to 10 mg/injection, and the patient is treated at intervals of every 14 days or so. During treatment, the patient is monitored for change in status of the cancer, typically by standard blood cell assays. The treatment may be carried out in combination with other cancer treatments, including drug or radio-isotope therapy, and may be continued until a desired improvement in patient condition is attained.

The immunotoxin is also useful in treating an autoimmune disease, such as multiple sclerosis, rheumatoid arthritis, and SLE. In this method, a patient diagnosed with an autoimmune disease is treated by administration of the immunotoxin, preferably administered by IV injection in a suitable physiological carrier. The antibody dose is preferably 1 to 10 mg/injection, and the patient is treated at intervals of every 14 days or so. During treatment, the patient is monitored for improvement in status, e.g., reduced level of pain or discomfort associated with the condition. The treatment may be carried out in combination with other treatments, such as treatment with immunosuppressive drugs, and may be continued until a desired improvement in patient condition is attained, or over an extended period to alleviate symptoms.

As can be appreciated from the studies above, the immunotoxin of the invention provides a number of advantages as a therapeutic agent specific against CD-19 expressing cells. The immunotoxin is highly specific against CD-19 expressing cells and is active at very low concentrations, e.g., in the nM range.

Due to the absence of the Fc portion, undesirable interactions of the Fc portion with Fc receptors on cells other than the tumor target cells are prevented.

The stable link between antibody-portion and toxin moiety leads to reduced non-specific toxicities due to the breakage of this bond in the extracellular space, and ensures that the toxin will be largely confined to target cells.

The following examples illustrate, but are in no way intended to limit the invention.

### Materials and Methods

A. Bacterial Strains and Plasmids

Escherichia coli XL1 -Blue (Stratagene, Amsterdam, the Netherlands) was used for the amplification of plasmids and cloning, and E. coli TG1 (from Dr. G. Winter, MRC, Cambridge, United Kingdom) for screening of antibody libraries. Libraries were generated in the phagemid vector pAK100, and pAK400 was used for the expression of soluble scFvs (Krebber, A., Bornhauser, S., Burmester, J., Honegger, A., Willuda, J., Bosshard, H. R., and Pluckthun, A. Reliable cloning of functional antibody variable domains from hybridomas and spleen cell repertoires employing a reengineered phage display system. J Immunol Methods, 201: 35-55, 1997). E. coli BL21 (DE3; Novagen, Inc., Madison, Wis.) served for the expression of scFv-ETA′ fusion protein.

B. Cell Lines

Leukemia-derived cell lines Nalm-6, Namalwa, Reh, CEM (DSMZ; German Collection of Microorganisms and Cell Lines, Braunschweig, Germany) and SEM (Greil, J., Gramatzki, M., Burger, R., Marschalek, R., Peltner, M., Trautmann, U., Hansen-Hagge, T. E. Bartram, C. E., Fey, G. H., Stehr, K. The acute lymphoblastic leukemia cell line SEM with t(4;11) chromosomal rearrangement is biphenotypic and responsive to interleukin-7. Br J Haematol, 86: 275-283, 1994) were cultured in RPMI 1640-Glutamax-I (Sigma, Deisenhofen, Germany) containing 10% FCS and penicillin and streptomycin (Invitrogen) at 100 units/ml and 100 μg/ml, respectively.

### EXAMPLE 1

### Preparation of CD-19 scFv Antibody

Total RNA was prepared from the hybridoma 4G7 (Meeker, T. C., Miller, R. A., Link, M. P., Bindl, J., Warnke, R., and Levy, R. A unique human B lymphocyte antigen defined by a monoclonal antibody, Hybridoma, 3: 305-320, 1984; Krebber, A., Bornhauser, S., Burmester, J., Honegger, A., Willuda, J., Bosshard, H. R., and Pluckthun, A. Reliable cloning of functional antibody variable domains from hybridomas and spleen cell repertoires employing a reengineered phage display system. J Immunol Methods, 201: 35-55, 1997). First-strand cDNA was prepared from 10-15 μg of total RNA (Krebber, A., Bornhauser, S., Burmester, J., Honegger, A., Willuda, J., Bosshard, H. R., and Pluckthun, A. Reliable cloning of functional antibody variable domains from hybridomas and spleen cell repertoires employing a reengineered phage display system. J Immunol Methods, 201: 35-55, 1997). PCR amplification of immunoglobulin variable region cDNAs and cloning into the phagemid vector pAK100 was performed as described (Krebber, A., Bornhauser, S., Burmester, J., Honegger, A., Willuda, J., Bosshard, H. R., and Pluckthun, A. Reliable cloning of functional antibody variable domains from hybridomas and spleen cell repertoires employing a reengineered phage display system. J Immunol Methods, 201: 35-55, 1997; Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001). Propagation of combinatorial scFv libraries and filamentous phages was performed by following published procedures (Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001).

A. Panning of Phage Display Libraries with Intact Cells

Panning of phage display libraries with intact cells was carried out as described (Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001) using CD19-positive SEM cells. Bound phages were eluted with 50 mM HCl.

B. Bacterial Expression and Purification of Soluble scFv Antibodies

For the soluble expression of antibody fragments, cDNA coding for the CD19-specific scFv was subcloned into the expression vector pAK400, and the plasmids were propagated in E. coli HB2151 (from Dr. G. Winter; MRC, Cambridge, United Kingdom). Expression and purification of CD19-specific scFv antibodies was performed as described (Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001).

### EXAMPLE 2

### Construction and Expression of scFv-ETA′ Fusion Protein

Sequences coding for the CD19-specific scFv were excised from the pAK400-anti CD19 expression construct and were cloned into the vector pASK/HisCD19ETA#3 (M. Peipp, unpublished data). The plasmid was digested with NcoI and NotI and ligated into the vector pet27b(+)-Strep-His-CD33-ETA′-KDEL (M. Schwemmlein, unpublished data), resulting in the vector pet27b(+)-STREP-His-CD19-ETA′-KDEL.

The scFv-ETA′ fusion protein was expressed under osmotic stress conditions as described (Barth, S., Huhn, M., Matthey, B., Tawadros, S., Schnell, R., Schinkothe, T., Diehl, V., and Engert, A. Ki-4(scFv)-ETA′, a new recombinant anti-CD30 immunotoxin with highly specific cytotoxic activity against disseminated Hodgkin tumors in SCID mice. Blood, 95: 3909-3914, 2000). Induced cultures were harvested 16-20 h after induction. The bacterial pellet from 1 liter culture was resuspended in 200 ml of periplasmatic extraction buffer [100 mM Tris, pH 8.0, 0.5 M sucrose, 1 mM EDTA] for 3 h at 4° C. The scFv-ETA′ fusion protein was enriched by affinity chromatography using streptactin agarose beads (IBA GmbH, Goettingen, Germany; Skerra, A. and Schmidt, T. G. Use of the Strep-Tag and streptavidin for detection and purification of recombinant proteins. Methods Enzymol, 326: 271-304, 2000) according to manufacturers instructions.

### EXAMPLE 3

### Characterization of scFv-ETA′ Immunotoxin

A. Immunotoxin Binding to Cells

The binding of scFvs to cells was analyzed using a FACSCalibur FACS instrument and CellQuest software (Becton Dickinson, Mountain View, Calif.). Cells were stained with scFv antibodies as described (Peipp, M., Simon, N., Loichinger, A., Baum, W., Mahr, K., Zunino, S. J., and Fey, G. H. An improved procedure for the generation of recombinant single-chain Fv antibody fragments reacting with human CD13 on intact cells. J Immunol Methods, 251: 161-176, 2001). A nonrelated scFv served as a control for background staining. Ten thousand events were collected for each sample, and analyses of whole cells were performed using appropriate scatter gates to exclude cellular debris and aggregates. To monitor binding of the scFv-ETA′ fusion protein, 5×10⁵ cells were incubated for 30 min on ice with 20 μl of the immunotoxin at a concentration of 5 μg/ml. A nonrelated immunotoxin served as a control for background staining. The cells were washed with PBA buffer [containing PBS, 0.1% BSA, and 7 mM Na-azide] and then incubated with 50 μl of a polyclonal rabbit anti-Pseudomonas ETA serum (Sigma) diluted 1:250 in PBA buffer. Cells were washed and incubated with fluorescein-iso-thiocyanate (FITC)-conjugated pig anti-rabbit-IgG (DAKO Diagnostica GmbH, Hamburg, Germany) for 30 min. After a final wash, cells were analyzed by FACS.

B. Measurement of Cytotoxic Effects of Immunotoxins

For dose response experiments, cells were seeded at 2.5×10⁵/ml in 24-well plates, and immunotoxin was added at varying concentrations. Cell death was measured by staining nuclei with a hypotonic solution of PI as described (Dorrie, J., Gerauer, H., Wachter, Y., and Zunino, S. J.). Resveratrol induces extensive apoptosis by depolarizing mitochondrial membranes and activating caspase-9 in acute lymphoblastic leukemia cells. Cancer Res, 61: 4731-4739, 2001; Nicoletti, I., Migliorati, G., Pagliacci, M. C., Grignani, F., and Riccardi, C. A rapid and simple method for measuring thymocyte apoptosis by propidium iodide staining and flow cytometry. J Immunol Methods, 139: 271-279, 1991). The extent of cell death was determined by measuring the fraction of nuclei with subdiploid DNA content. Fifteen thousand events were collected for each sample and analyzed for subdiploid nuclear DNA content. To determine whether cell death was attributable to apoptosis, cells were seeded at 2.5×10⁵/ml and treated with the immunotoxin. Whole cells were stained with FITC-conjugated Annexin V (Pharmingen, Heidelberg, Germany; Vermes, I., Haanen, C., Steffens-Nakken, H., and Reutelingsperger, C. A novel assay for apoptosis. Flow cytometric detection of phosphatidylserine expression on early apoptotic cells using fluorescein labelled Annexin V. J Immunol Methods, 184: 39-51, 1995) and PI in PBS according to the manufacturer's protocol. For blocking experiments, a 20-fold molar excess of the parental CD19 antibody 4G7 was added to the culture 1 h before adding the immunotoxin. For determination of viable cells, cells were stained by trypan blue and counted.

Although the invention has been described with respect to specific embodiments and applications, it will be appreciated that various changes and modification may be made within the spirit of the invention.

Sequence Listing:

SEQ ID NO: 1, polynucleotide sequence of the antibody-toxin conjugate;

SEQ ID NO: 2, amino acid sequence of the antibody-toxin conjugate;

SEQ ID NO: 3, amino acid sequence of the modified ETA′ protein;

SEQ ID NO: 4, amino acid sequence of the linker coupling the variable-light and variable-heavy chains of the scFv antibody;

SEQ ID NO: 5, amino acid sequence of the linker coupling the scFv antibody to the modified ETA′ toxin;

SEQ ID NO: 6, sequence that promotes transport of a protein to the endoplasmic reticulum;

SEQ ID NO: 7, sequence that promotes transport of a protein to the endoplasmic reticulum; and

SEQ ID NO: 8, STREP tag.

SEQ ID NO: 1
tggagccacccgcagttcgaaaaaatcgaagggcgccatcaccatcaccatcacggggcccagccggccat
ggcggactacaaagatattgtgatgacccaggctgcaccctctatacctgtcactcctggagagtcagtatccat
ctcctgcaggtctagtaagagtctcctgaatagtaatggcaacacttacttgtattggttcctgcagaggccaggc
cagtctcctcagctcctgatatatcggatgtccaaccttgcctcaggagtcccagacaggttcagtggcagtgggt
caggaactgctttcacactgagaatcagtagagtggaggctgaggatgtgggtgtttattactgtatgcaacatct
agaatatccgctcacgttcggtgctgggcaccaagctggaaatcaaacgtggtggtggtggttctggtggtggtgg
ttctggcggcggcggctccagtggtggtggatcccaggttcagcttcagcagtctggacctgagctgataaagc
ctggggcttcagtgaagatgtcctgcaaggcttctggatacacattcactagctatgttatgcactgggtgaagca
gaagcctgggcagggccttgagtggattggatatattaatccttacaatgatggtactaagtacaatgagaagttc
aaaggcaaggccacactgacttcagacaaatcctccagcacagcctacatggagctcagcagcctgacctct
gaggactctgcggtctattactgtgcaagagggacttattactacggtagtagggtatttgactactggggccaag
gcaccactctcacagtcaccgtctcctcggcctcgggggccggtggtggcggcagtggtggtggcggcagtgg
tggtggcggcagtggtggtggcggcagtgcggccgcgctagagggcggcagcctggccgcgctgaccgcgc
accaggcctgccacctgccgctggagactttcacccgtcatcgccagccgcgcggctgggaacaactggagc
agtgcggctatccggtgcagcggctggtcgccctctacctggcggcgcgactgtcatggaaccaggtcgacca
ggtgatccgcaacgccctggccagccccggcagcggcggcgacctgggcgaagcgatccgcgagcagcc
ggagcaggcccgtctggccctgaccctggccgccgccgagagcgagcgcttcgtccggcagggcaccggc
aacgacgaggccggcgcggtccagcgccgacgtggtgagcctgacctgcccggtcgccgccggtgaatgcg
cgggcccggcggacagcggcgacgccctgctggagcgcaactatcccactggcgcggagttcctcggcgac
ggtggcgacgtcagcttcagcacccgcggcacgcagaactggacggtggagcggctgctccaggcgcacc
gccaactggaggagcgcggctatgtgttcgtcggctaccacggcaccttcctcgaagcggcgcaaagcatcgt
cttcggcggggtgcgcgcgcgcagccaggatctcgacgcgatctggcgcggtttctatatcgccggcgatccg
gcgctggcctacggctacgcccaggaccaggaacccgacgcgcgcggccggatccgcaacggtgccctgc
tgcgggtctatgtgccgcgctcgagcctgccgggcttctaccgcaccggcctgaccctggccgcgccggaggc
ggcgggcgaggtcgaacggctgatcggccatccgctgccgctgcgcctggacgccatcaccggccccgagg
aggaaggcgggcgcctggagaccattctcggctggccgctggccgagcgcaccgtggtgattccctcggcga
tccccaccgacccgcgcaacgtcggcggcgacctcgacccgtccagcatccccgacaaggaacaggcgat
cagcgccctgccggactacgccagccagcccggcaaaccgccgaaggacgagctg

SEQ ID NO: 2.
WSHPQFEKIEGRHHHHHHGAQPAMADYKDIVMTQAAPSIPVTPGESVSISCRSSKSLLN
SNGNTYLYWFLQRPGQSPQLLIYRMSNLASGVPDRFSGSGSGTAFTLRISRVEAEDVGV
YYCMQHLEYPLTFGAGTKLEIKRGGGGSGGGGSGGGGSSGGGSQVQLQQSGPELIKP
GASVKMSCKASGYTFTSYVMHWVKQKPGQGLEWIGYINPYNDGTKYNEKFKGKATLTS
DKSSSTAYMELSSLTSEDSAVYYCARGTYYYGSRVFDYWGQGTTLTVTVSSASGAGGG
GSGGGGSGGGGSGGGGSAAALEGGSLAALTAHQACHLPLETFTRHRQPRGWEQLEQ
CGYPVQRLVALYLAARLSWNQVDQVIRNALASPGSGGDLGEAIREQPEQARLALTLAAA
ESERFVRQGTGNDEAGAASADVVSLTCPVAAGECAGPADSGDALLERNYPTGAEFLGD
GGDVSFSTRGTQNWTVERLLQAHRQLEERGYVFVGYHGTFLEAAQSIVFGGVRARSQD
LDAIWRGFYIAGDPALAYGYAQDQEPDARGRIRNGALLRVYVPRSSLPGFYRTGLTLAAP
EAAGEVERLIGHPLPLRLDAITGPEEEGGRLETILGWPLAERTVVIPSAIPTDPRNVGGDL
DPSSIPDKEQAISALPDYASQPGKPPKDEL

SEQ ID NO: 3:
EGGSLAALTAHQACHLPLETFTRHRQPRGWEQLEQCGYPVQRLVALYLAARLSWNQVD
QVIRNALASPGSGGDLGEAIREQPEQARLALTLAAAESERFVRQGTGNDEAGAASADVV
SLTCPVAAGECAGPADSGDALLERNYPTGAEFLGDGGDVSFSTRGTQNWTVERLLQAH
RQLEERGYVFVGYHGTFLEAAQSIVFGGVRARSQDLDAIWRGFYIAGDPALAYGYAQDQ
EPDARGRIRNGALLRVYVPRSSLPGFYRTGLTLAAPEAAGEVERLIGHPLPLRLDAITGPE
EEGGRLETILGWPLAERTVVIPSAIPTDPRNVGGDLDPSSIPDKEQAISALPDYASQPGKP
PKDEL

SEQ ID NO: 4
GGGGSGGGGSGGGGSSGGGS

SEQ ID NO: 5
GGGGSGGGGSGGGGSGGGGS

SEQ ID NO: 6
KDEL

SEQ ID NO: 7
REDLK

SEQ ID NO: 8
WSHPQFEK

1. An immunotoxin for use in treating a subject having a cancer associated with malignant B-lineage cells, such as chronic lymphocytic leukemia, Non-Hodgkin lymphoma, and acute lymphoblastic leukemia, comprising
(a) an anti-CD19 antibody lacking an Fc fragment,
(b) a modified exotoxin A protein having both Domains II and III, but lacking Domain I, and
(c) a peptide linker joining the C-terminal end of the antibody to the N-terminal end of the modified exotoxin A protein, said linker being substantially resistant to extracellular cleavage.

2. The immunotoxin of claim 1, wherein said modified exotoxin A protein has a C-terminal KDEL sequence (SEQ ID NO: 6) that promotes transport of the protein to the endoplasmic reticulum of cells that have taken up the immunotoxin.

3. The immunotoxin of claim 1, wherein the modified exotoxin A protein has the sequence identified by SEQ ID NO: 3.

4. The immunotoxin of claim 1, wherein said antibody is a single-chain scFv antibody composed of a variable-region light chain coupled to a variable-region heavy chain through a glycine/serine peptide linker.

5. The immunotoxin of claim 1, wherein the antibody is coupled to the modified exotoxin protein through a glycine/serine peptide linker.

6. The immunotoxin of claim 5, wherein the linker coupling the antibody to the modified exotoxin protein has the sequence identified as SEQ ID NO: 5.

7. A method of treating a subject having a cancer associated with malignant B-lineage cells, such as chronic lymphocytic leukemia, Non-Hodgkin lymphoma, and acute lymphoblastic leukemia, comprising administering to the patient, a therapeutically effective amount of an immunotoxin composed of:
(a) an anti-CD19 antibody lacking an Fc fragment,
(b) a modified exotoxin A protein having both Domains II and III, but lacking Domain I, and
(c) a peptide linker joining the C-terminal end of the antibody to the N-terminal end of the modified exotoxin A protein, said linker being substantially resistant to extracellular cleavage.

8. The method of claim 7, wherein said modified exotoxin A protein in the immunotoxin administered has a C-terminal KDEL sequence (SEQ ID NO: 6) that promotes transport of the protein to the endoplasmic reticulum within cells that have taken up the immunotoxin.

9. The method of claim 8, wherein the modified exotoxin A protein in the immunotoxin administered has the sequence identified by SEQ ID NO: 3.

10. The method of claim 7, wherein said antibody is a single-chain scFv antibody composed of a variable-region light chain coupled to a variable-region heavy chain through a glycine/serine peptide linker.

11. The method of claim 10, wherein the antibody in the immunotoxin administered is coupled to the modified exotoxin protein through a glycine/serine peptide linker.

12. The method of claim 11, wherein the linker coupling the antibody to the modified exotoxin protein in the immunotoxin administered has the sequence identified as SEQ ID NO: 5.

13. (canceled)

14. A method for delivering exotoxin A (ETA) to a human subject, in the treatment of a cancer having cancer-specific cell-surface antigens, comprising
(a) replacing Domain I of the ETA with a single-chain antibody specific against the cell-surface antigen and a peptide linker joining the C-terminal end of the antibody to the N-terminal end of the modified ETA, said linker being substantially resistant to extracellular cleavage, and
(b) replacing the REDLK C-terminal sequence (SEQ ID NO: 7) of ETA with a KDEL sequence (SEQ ID NO: 6) that promotes transport of the protein to endplasmic reticulum.

15. The method of claim 14, for treating a subject having a cancer associated with malignant B-lineage cells, such as chronic lymphocytic leukemia, Non-Hodgkin lymphoma, and acute lymphoblastic leukemia, wherein the single-chain antibody is specific against CD19 B-cell antigen.

16. The method of claim 14, wherein said linker includes a glycine/serine peptide linker.

17. The method of claim 16, wherein said linker has the sequence identified as SEQ ID NO: 5.


SEQUENCE LISTING (8 sequences)

SEQ ID NO: 1 - DNA, 2019 bp, Artificial Sequence
  sequence encoding synthetic fusion protein
tggagccacccgcagttcgaaaaaatcgaagggcgccatcaccatcaccatcacggggcccagccggccatggcggactacaaagatattgtgatgacccaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgctcacgttcggtgctgggaccaagctggaaatcaaacgtggtggtggtggttctggtggtggtggttctggcggcggcggctccagtggtggtggatcccaggttcagcttcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtcaccgtctcctcggcctcgggggccggtggtggcggcagtggtggtggcggcagtggtggtggcggcagtggtggtggcggcagtgcggccgcgctagagggcggcagcctggccgcgctgaccgcgcaccaggcctgccacctgccgctggagactttcacccgtcatcgccagccgcgcggctgggaacaactggagcagtgcggctatccggtgcagcggctggtcgccctctacctggcggcgcgactgtcatggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccagcgccgacgtggtgagcctgacctgcccggtcgccgccggtgaatgcgcgggcccggcggacagcggcgacgccctgctggagcgcaactatcccactggcgcggagttcctcggcgacggtggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggatctcgacgcgatctggcgcggtttctatatcgccggcgatccggcgctggcctacggctacgcccaggaccaggaacccgacgcgcgcggccggatccgcaacggtgccctgctgcgggtctatgtgccgcgctcgagcctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgaaggacgagctg

SEQ ID NO: 2 - protein, 673 aa, Artificial Sequence
  synthetic fusion protein
WSHPQFEKIEGRHHHHHHGAQPAMADYKDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPLTFGAGTKLEIKRGGGGSGGGGSGGGGSSGGGSQVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVTVSSASGAGGGGSGGGGSGGGGSGGGGSAAALEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 3 - protein, 361 aa, Artificial Sequence
  modified exotoxin A sequence
EGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 4 - protein, 20 aa, Artificial Sequence
  synthetic linker
GGGGSGGGGSGGGGSSGGGS

SEQ ID NO: 5 - protein, 20 aa, Artificial Sequence
  synthetic linker
GGGGSGGGGSGGGGSGGGGS

SEQ ID NO: 6 - protein, 4 aa, Artificial Sequence
  synthetic transport sequence
KDEL

SEQ ID NO: 7 - protein, 5 aa, Artificial Sequence
  synthetic transport sequence
REDLK

SEQ ID NO: 8 - protein, 8 aa, Artificial Sequence
  synthetic strep tag
WSHPQFEK